(19) United States
(12) Patent Application Publication
(10) Pub. No.: US 2013/0005803 A1
(43) Pub. Date: Jan. 3, 2013

(54) USE OF COMPOUNDS IN THE TREATMENT OR PREVENTION OF SKIN DISORDERS

(75) Inventor: Jérôme Aubert, Grasse (FR)

(73) Assignee: GALDERMA RESEARCH & DEVELOPMENT, Biot (FR)

(21) Appl. No.: 13/516,801

(22) PCT Filed: Dec. 15, 2010
(86) PCT No.: PCT/EP2010/069739

§ 371 Date: Sep. 4, 2012

Related U.S. Application Data
(60) Provisional application No. 61/282,109, filed on Dec. 17, 2009.

(51) Int. Cl. A61K 31/17; A61K 31/277; A61P 17/00; A61P 17/10; C07C 275/40; C07C 275/34

(52) U.S. Cl. 514/521; 564/50; 514/597; 558/417

(57) ABSTRACT

The use of compounds in the treatment of skin disorders is described. In particular, the use of a compound of formula (I)

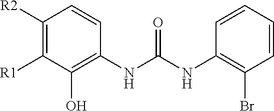

or one of its pharmaceutically acceptable salts, pharmaceutically acceptable solvates or hydrates in the preparation of a medicament to treat inflammatory skin pathologies is described.

The present invention relates to a novel use of a compound of the formula (I) or of one of its pharmaceutically acceptable salts, pharmaceutically acceptable solvates, or hydrates, in the preparation of a medicament intended to prevent and/or treat skin disorders and preferably acne, rosacea and neutrophilic dermatosis.

Acne vulgaris is the most common skin condition affecting millions of people worldwide, it's a chronic inflammatory disease of the pilosebaceous units characterized by the formation of comedones, erythrematous papules and pustules, less frequently by nodules or pseudocyts (Strauss J S, Pochi P E, Downing D T: Acne: perspectives. J Invest Dermatol 1974, 62:321-325]. It is a pleomorphic disorder with multifactorial pathogenesis; and still incompletely understood (Oberemok S S, Shalita A R: Acne vulgaris, I: pathogenesis and diagnosis. Cutis 2002, 70:101-105).

The inflammatory stage of acne vulgaris is usually of greatest concern for the patient. A number of morphologically different inflammatory lesions may be formed that can be painful and unsightly. In 30% of patients, such lesions lead to scarring. Inflammatory acne and acne scarring can have significant psychological effects on patients, including depression, anxiety, and poor self-image especially during the emotionally critical period of adolescence (Aktan S, Ozmen E, Sanli B: Anxiety, Depression, and Nature of Acne Vulgaris in Adolescents. Int J Dermatol 2000, 39:354-357). Recent evidence supports a pivotal role for cellular inflammatory events at all stages of acne lesion development. Inflammatory acne lesions may be summarized by a succession of events: the normal sebaceous gland follicle develops into microcomedone and comedone through keratinocyte hyperproliferation and reduced sloughing. This process may associate with release of early inflammatory cytokines such as IL-1a we assumed that. P. Acnes population density increases (STEP 1). Initiation of inflammation may occur through either a specific CD4+ T-cell-mediated pathway or a non-specific pathway involving increased production of proinflammatory chemokines and cytokines by keratinocytes (STEP 2). Inflammation is increased by activation of neutrophils (STEP 3). Neutrophils release lysosomal enzymes, lipases, hyaluronidases, oxygenated agents and a large panel of pro-inflammatory stimuli that lead to rupture of follicular epithelium and further inflammation. This last step participates actively to the mechanism of scar formation (STEP 4) (For a review, see ACNE: INFLAMMATION; Farra and Ingham; Clinics and Dermatology; 2005).

IL-8 (CXCL-8) is a member of the CXC chemokine family that plays a primordial role in the trafficking of neutrophils to the site of inflammation (For a review see Busch-Petersen J.; Curr Top Med. Chem. 2006; 6(13):1345-52).

Elevated levels of IL-8 and related chemokines (CXCL2) have been reported in inflammatory acne lesions. Both CXCR1 and CXCR2 mRNA were detected in non lesional and lesional skin (Trivedi N R et al. J Invest Dermatol. 2006 May; 126(5):1071-9;

Two chemokine G-Protein-coupled, seven-transmembrane receptors (CXCR1 and CXCR2) are known to be specifically activated by IL-8. While CXCR2 binds with high affinity to IL-8 and other chemokines such as CXCL6, CXCL5, CXCL2 and CXCL1, CXCR1 is less promiscuous and binds only IL-8. In addition to neutrophil chemotaxis and activation, these receptors are involved in the chemotaxis and activation of monocytes, macrophages and subsets of T-cells. IL-8 may, also, directly participate to the hyperproliferation of keratinocytes and neo-vascularisation.

Therefore, potent dual antagonists of CXCR1 and CXCR2 may hold promise in limiting the deleterious effects of the inflammatory response and angiogenesis mediated by IL-8 and associated chemokines, and thereby prevent scarring.

In prio art, particularly N,N′ Diarylurea compounds as described by Winddowson et al. in J. Med. Chem. 2004, 47, 1319-1321 were evaluated as antagonists for CXCR2. Those small molecules were found active both in vitro against human neutrophils and in vivo in rabbit models of ear oedema and neutropenia.

Patent application WO00/35442 disclosed hydroxyl urea sulfonamides as IL-8 antagonistes and used for treatment of diseases mediated by that chemokine such as atopic dermataitis, osteoarthritis, pulmonary diseases or disorders, acne.

Patent Application WO 02/079122 describes a family of diarylurea derivatives in which compound of formula (I) is disclosed as well as a chemical process in order to increase metabolic stability and half-life.

It is now found, unexpectedly, that some of the compounds of this patent application are CXCR1 and CXCR2 antagonists and capable of being active in preventing and/or treating skin disorders and preferably acne and rosacea.

The present invention thus relates to the use of compound of following formula (I):

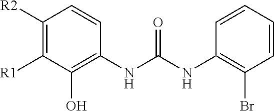

Wherein R1 is selected from hydrogen, bromide, alkyl having from 1 to 4 carbon atomes and preferably chosen from methyl and ethyl; cyanide, chloride and R2 is selected from cyanide or nitro;

its pharmaceutically acceptable salts, its pharmaceutically acceptable solvates or their hydrates, in the preparation of a medicament intended to prevent and/or treat skin disorders. These pathologies are keratinic disorders in particular all forms of acne and neutrophilic dermatosis.

In a preferred embodiment, the present invention relates to the use of compound of formula (I) wherein R1 is selected from bromide or hydrogen and R2 is selected from cyanide or nitro.

The invention relates more specifically to the use of N-(3-bromo-4-cyano-2-hydroxyphenyl)-W-(2-bromophenyl)urea also known as SB656933 or the use of (1-(2-bromo-phenyl)-3-(2-hydroxy-4-nitro-phenyl)-urea) also known as SB 225002 or of one of their pharmaceutically acceptable salts, pharmaceutically acceptable solvates or hydrates in the preparation of a medicament intended to prevent and/or treat keratinic disorders and preferably acne and neutrophilis dermatosis.

In alternative embodiment of invention, the present invention provides a method for preventing and/or treating skin disorders and preferably acne, rosacea and neutrophilic dermatosis wherein at least one compound chosen from the compounds of formula (I):

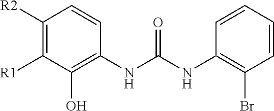

Wherein R1 is selected from hydrogen, bromide, alkyl having from 1 to 4 carbon atomes and preferably chosen from methyl and ethyl; cyanide, chloride and R2 is selected from cyanide or nitro; or its pharmaceutically acceptable salts, their pharmaceutically acceptable solvates and their hydrates, is administered to an individual in need thereof.

In a preferred embodiment, the present invention provides compound of formula (I) wherein R1 is selected from bromide or hydrogen and R2 is selected from cyanide or nitro. In a specific embodiment, the present method provides compounds chosen from N-(3-bromo-4-cyano-2-hydroxyphenyl)-M-(2-bromophenyl)urea also known as SB656933 or (1-(2-bromo-phenyl)-3-(2-hydroxy-4-nitro-phenyl)-urea) also known as SB 225002 or of one of their pharmaceutically acceptable salts, pharmaceutically acceptable solvates or hydrates.

The salts of the compounds of formula (I) according to the invention comprise salts with organic or inorganic bases, for example alkali metal salts, such as lithium, sodium or potassium salts.

The term “hydrate of a compound of formula (I)” is understood to mean a combination of this compound with one or more water molecules.

The term “solvate of a compound of formula (I)” is understood to mean the combination resulting from the attachment of a solvent to the crystals of compound of formula (I) which are formed in the presence of this solvent.

For their use as medicament, the compound of formula (I), their pharmaceutically acceptable salts, their pharmaceutically acceptable solvates or their hydrates have to be formulated as a pharmaceutical composition, preferably a dermatological composition.

Another subject-matter of the present invention is thus pharmaceutical compositions, in particular dermatological compositions, comprising at least one compound chosen from the compound of formula (I).

Such compositions can be intended, and thus appropriate, for oral, topical, enteral, parenteral, ocular, sublingual, inhaled, subcutaneous, intramuscular, intravenous, transdermal or rectal administration. The compound of formula (I), optionally in the form of a salt, solvate and/or hydrate which is pharmaceutically acceptable, alone or in combination with another active principle, can be administered in a unit of administration form, as a mixture with conventional pharmaceutical carriers or excipients, to animals and human beings. Preferably, the pharmaceutical composition is packaged in a form suitable for oral or topical administration.

The compositions according to the invention comprise at the compound of formula (I) or one of its pharmaceutically acceptable salts, pharmaceutically acceptable solvates or hydrates in amounts sufficient to obtain the desired prophylactic or therapeutic effect. The useful dosage varies according to the age, sex and weight of the patient.

The compound of formula (I) or one of its salts, solvates or hydrates will preferably be administered in a proportion of 0.01 to 100 mg/kg and per day, advantageously of 0.01 to 50 mg/kg and per day. It is also possible to administer such doses in 2 to 4 daily administrations. Although these dosages are examples of average situations, there may be specific cases where higher or lower dosages are appropriate; such dosages also come within the invention.

The compositions according to the invention comprise a physiologically acceptable carrier or at least one pharmaceutically acceptable excipient chosen according to the pharmaceutical form, in particular dermatological form, desired and the method of administration chosen.

The term “physiologically acceptable carrier” and the term “pharmaceutically acceptable excipient” are understood to mean respectively a carrier and an excipient which are compatible with the skin, mucous membranes and superficial body growths.

For oral administration, the pharmaceutical or dermatological composition can be provided in the form of tablets, including sugar-coated tablets, hard gelatin capsules, pills, syrups, suspensions, solutions, powders, granules, emulsions, capsules or microspheres or nanospheres or lipid or polymeric vesicles which make possible controlled release.

Parenterally, the composition can be provided in the form of solutions or suspensions for infusion or for injection.

To obtain a solid composition for oral administration, the active principle can be mixed with at least one inert diluent, such as sucrose, lactose or starch. Generally, other additives, such as a lubricant, for example magnesium stearate, can be added. In the case of capsules, tablets or pills in particular, a buffer can be added. In the case of liquid oral compositions, an inert diluent, such as water, can be used.

Topically, the pharmaceutical composition according to the invention is more particularly intended for the treatment of the skin and mucous membranes and can be provided in the form of ointments, creams, milks, salves, powders, impregnated pads, syndets, solutions, gels, sprays, foams, suspensions, lotions, sticks, shampoos or washing bases. They can also be provided in the form of suspensions of microspheres or nanospheres or lipid or polymeric vesicles or of polymeric patches and of hydrogels which make possible controlled release. This topical composition can be provided in the anhydrous form, in the aqueous form or in the form of an emulsion.

The compound of formula (I) or one of its salts, solvates or hydrates, when it is administered orally, is administered in a proportion of 0.01 to 100 mg/kg and per day, advantageously of 0.01 to 50 mg/kg.

The compound of formula (I) or one of its salts, solvates or hydrates, when it is administered topically, is used at a concentration generally of between 0.001 and 10% by weight, preferably between 0.01 and 5% by weight, with respect to the total weight of the composition.

The pharmaceutical and dermatological compositions as described above can thus comprise inert additives or even pharmacodynamically active additives or combinations of these additives, and in particular:
wetting agent;
flavour enhancers;
preservatives, such as para-hydroxybenzoic acid esters;
stabilizing agents;
moisture regulators;
pH regulators;
osmotic pressure modifiers;
emulsifying agents;
UV-A and UV-B screening agents;
antioxidants, such as α-tocopherol, butylated hydroxyanisole or butylated hydroxytoluene, superoxide dismutase, ubiquinol or certain metal-chelating agents;
emollients;
moisturizing agents, such as glycerol, PEG 400, thiamorpholinone and its derivatives or urea;

Of course, a person skilled in the art will take care to choose the optional compound or compounds to be added to these compositions so that the desired effect on psoriasis or eczema is not, or not substantially, detrimentally affected by the envisaged addition.

The compounds of formula (I) is in particular synthesized as described in Application WO 02/079122.

The study of the properties of the compound of formula (I) has shown that the compound of formula (I) and its pharmaceutically acceptable salts, solvates or hydrates are not toxic and have an antiinflammatory activity in the treatment of acne, neutrophilic dermatosis in its largest sense, sweet syndrome, eccrine hydranite, SAPHO syndrome, Sneddon Wilkinson syndrome (sub-corneal pustulosis) pyoderma gangrenosum, erythema elevatum duitinum, psoriasis, psoriasis vulgaris, pustular psoriasis, palmo-plantar pustulosis, exanthematic pustulosis (PEAG), pustular vascularitis, child acro-pustulosis, Behcet disease as well as certain auto-immunebullous disease such as herpes derived form of dermatite, IgA neutrophilic dermatosis, IgA intra-epiderm pustilosis, bulleous pemphigoide, IgA pemphigus, vascularitis, Fiellinger Leroy Reiter, syndrome, scalp pustulosis, Hallopeau continous acrodermatite and dermatosis associated with angio-immunoblastic lymphodenopathy, with dysmielopoësis induced by cyclophosphamide, with p-ANCA antibodies, pilotrope purpuric fungal mycosis.

Rosacea is a common, but often overlooked, skin condition of uncertain etiology that can lead to significant facial disfigurement, ocular complications, and severe emotional distress. A reviewed standard classification system for rosacea was published in the June 2004 issue of the Journal of the American Academy of Dermatology. Developed by the National Rosacea Society Expert Committee on the Classification and Staging of Rosacea and reviewed by rosacea experts worldwide, it describes primary and secondary features of rosacea (as described below) and recognizes 4 patterns of signs and symptoms, designated as subtypes:

### Subtype 1: Erythematotelangiectatic Rosacea.

Subtype 1 is characterized by flushing and persistent central facial erythema. Telangiectases are common but not essential for the diagnosis.

### Subtype 2: Papulopustular Rosacea.

Subtype 2 includes persistent central facial erythema with transient papules, pustules, or both in a central facial distribution. Burning and stinging may also be reported.

### Subtype 3: Phymatous Rosacea.

This subtype may include thickening skin, irregular surface nodularities, and enlargement. Phymatous rosacea occurs most commonly as rhinophyma but may appear elsewhere, including the chin, forehead, cheeks, and ears. Patulous, expressive follicles may appear in the phymatous area, and telangiectases may be present.

### Subtype 4: Ocular Rosacea.

Ocular rosacea may include watery or bloodshot appearance (interpalpebral conjunctival hyperemia), foreignbody sensation, burning or stinging, dryness, itching, light sensitivity, blurred vision, telangiectasia of the conjunctiva and lid margin, or lid and periocular erythema. Blepharitis, conjunctivitis, and irregularity of the eyelid margins also may occur. Meibomian gland dysfunction presenting as chalazion, or chronic infection as manifested by hordeolum (stye), are common. Some patients may experience loss of vision as a result of corneal complications (punctate keratitis, corneal infiltrates, ulcers, or marginal keratitis). An ophthalmologic consultative approach to treatment may be required.

Central facial erythema and telangiectasis are predominant in the early stages of rosacea. This progresses to a chronic inflammatory infiltrate with central facial papules and pustules. Intermittent or chronic facial edema may also occur. Some patients develop rhinophyma, a coarse hypertrophy of the connective tissue and sebaceous glands of the nose evident as small knobby bumps on the nose.

The predominant, presenting complaints of rosacea are intermittent, central facial flushing and erythema. Itching is typically absent; however, many patients complain of a stinging pain (which can be severe) associated with flushing episodes. These flushing episodes are often socially embarrassing and can occur unpredictably or can be linked to environmental, chemical, food, or emotional triggers. Common triggers include exposure to the sun, cold weather, sudden emotion (laughter or embarrassment), hot beverages, and alcohol consumption.

Compound of formula (I) have been shown to have antagonist properties regarding CXCR1 and/or CXCR2 receptors.

For acne treatment and neutrophilic dermatosis, specific antagonist regarding CXCR1 or CXCR2 as well as dual antagonists are preferred.

For treating rosacea, specific antagonist regarding CXCR1 and dual antagonists are preferred.

Other characteristics, aspects, objectives and advantages of the invention will become still more clearly apparent on reading the description which follows and various concrete, but in no way limiting, examples intended to illustrate it.

### EXAMPLE 1

### Compositions

### A—Orally

0.2 g tablet

| | |
|---|---|
| Compound of formula (I) | 0.001 g |
| Starch | 0.114 g |
| Dicalcium phosphate | 0.020 g |
| Silica | 0.020 g |
| Lactose | 0.030 g |
| Talc | 0.010 g |
| Magnesium stearate | 0.005 g |

### B—Topically

### (a) Ointment

| | |
|---|---|
| Compound of formula (I) | 0.30 g |
| White petrolatum, pharmaceutical grade | q.s. for 100 g |

### (b) Lotion

| | |
|---|---|
| Compound of formula (I) | 0.10 g |
| Polyethylene glycol (PEG 400) | 69.90 g |
| 95% Ethanol | 30.00 g |

1. A method of preparing a medicament, the method comprising preparing the medicament with an effective amount of at least one compound of formula (I): 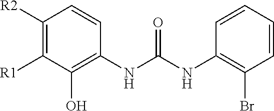
wherein R1 is selected from the group consisting of hydrogen, bromide, alkyl having from 1 to 4 carbon atoms; cyanide, and chloride and R2 is selected from cyanide or nitro;
or a pharmaceutically acceptable salt thereof or its pharmaceutically acceptable solvate or hydrate, and wherein the medicament is effective to treat a skin disorder.

2. The method as defined by claim 1, wherein the skin disorder is selected from the group consisting of acne, rosacea and neutrophilic dermatosis.

3. The method as defined by claim 1, wherein R1 is bromide or hydrogen and R2 is cyanide or nitro.

4. The method as defined by claim 1, wherein the compound of formula (I) is N-(3-bromo-4-cyano-2-hydroxyphenyl)-N′-(2-bromophenyl)urea; a pharmaceutically acceptable salt thereof or its pharmaceutically acceptable solvate or hydrate.

5. The method as defined by claim 3, wherein the compound of formula (I) is (1-(2-bromo-phenyl)-3-(2-hydroxy-4-nitro-phenyl)-urea); a pharmaceutically acceptable salt thereof or its pharmaceutically acceptable solvate or hydrate.

6. The method as defined by claim 1, wherein the compound is a medicinal product formulated for topical administration.

7. A method for treating a skin disorder, the method comprising administering an effective amount of at least one compound of formula (I): 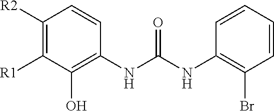
wherein R1 is selected from the group consisting of hydrogen, bromide, alkyl having from 1 to 4 carbon atoms; cyanide, and chloride and R2 is cyanide or nitro; or a pharmaceutically acceptable salt thereof or its pharmaceutically acceptable solvate or hydrate to an individual in need thereof.

8. The method as defined by claim 7, wherein the skin disorder is selected from the group consisting of acne, rosacea and neutrophilic dermatosis.

9. The method as defined by claim 1, wherein when R1 is an alkyl having from 1 to 4 carbon atoms, R1 is methyl or ethyl.

10. The method as defined by claim 7, wherein when R1 is an alkyl having from 1 to 4 carbon atoms R1 is methyl or ethyl.

11. The method as defined by claim 7, wherein R1 is bromide or hydrogen and R2 is cyanide or nitro.

12. The method as defined by claim 7, wherein the compound of formula (I) is N-(3-bromo-4-cyano-2-hydroxyphenyl)-N′-(2-bromophenyl)urea; a pharmaceutically acceptable salt thereof, or its pharmaceutically acceptable solvate or hydrate.

13. The method as defined by claim 7, wherein the compound of formula (I) is (1-(2-bromo-phenyl)-3-(2-hydroxy-4-nitro-phenyl)-urea); a pharmaceutically acceptable salt thereof or its pharmaceutically acceptable solvate or hydrate.

14. The method as defined by claim 7, wherein the compound is a medicinal product formulated for topical administration.
